(19) United States
(12) Patent Application Publication
(10) Pub. No.: US 2022/0016054 A1
(43) Pub. Date: Jan. 20, 2022

(54) EPINEPHRINE PARENTERAL FORMULATIONS

(71) Applicant: Somerset Therapeutics LLC, Hollywood, FL (US)

(72) Inventors: Mandar V. Shah, Somerset, NJ (US); Santhosh Kamath, Nelamangala (IN); Sathiyamoorthi Natarajan, Nelamangala (IN); Ilango Subramanian, Somerset, NJ (US); Veerappan Subramanian, Somerset, NJ (US)

(73) Assignee: Somerset Therapeutics LLC, Hollywood, FL (US)

(21) Appl. No.: 17/378,089

(22) Filed: Jul. 16, 2021

Related U.S. Application Data
(60) Provisional application No. 63/054,164, filed on Jul. 20, 2020.

(51) Int. Cl. A61K 31/137; A61K 47/02; A61K 47/12; A61K 47/18; A61K 9/08; A61K 9/00; A61K 47/20; A61K 47/22

(52) U.S. Cl. CPC A61K 31/137; A61K 47/02; A61K 47/12; A61K 47/22; A61K 9/08; A61K 9/0019; A61K 47/20; A61K 47/183

(57) ABSTRACT

The present invention relates to liquid formulations of epinephrine or a pharmaceutically acceptable salt thereof intended for parenteral administration. In particular, the invention provides liquid formulations comprising levorotatory-epinephrine (l-epinephrine) in a concentration of about 0.5 to about 1.5 mg/mL and at least one antioxidant. The antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination. The formulations prepared by using the current invention exhibit good physical and chemical stability.

### FIELD OF THE INVENTION

Disclosed herein are liquid formulations comprising epinephrine or a pharmaceutically acceptable salt thereof, particularly a stabilized liquid formulation comprising levorotatory-epinephrine (1-epinephrine) in a concentration of about 0.5 to 1.5 mg/mL and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination. The present invention also relates to a process of preparing such compositions and use thereof.

### BACKGROUND OF THE INVENTION

Epinephrine, or (−)-3,4-Dihydroxy-(methylamino) methylbenzyl alcohol, commonly known as adrenaline, is an endogenous adrenergic neurotransmitter synthesized and stored in the adrenal medulla. It is a polar compound characterized structurally by a catechol and an amine, and it is commonly available in a salt form. Epinephrine is water soluble and interacts in a variety of ways, depending on the type of receptor areas of target cells. The molecular weight of epinephrine is 183.2, with the following chemical structure:

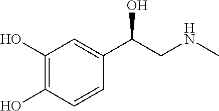

Epinephrine is a sympathomimetic drug that acts on both alpha- and beta-adrenergic receptors found ubiquitously throughout much of the body and can work rapidly to improve breathing, stimulate the heart, raise dropping blood pressure, reverse hives, and reduce swelling of the face, lips, and throat. Uses for epinephrine include emergency treatment of allergic reactions (Type 1), including anaphylaxis, induction and maintenance of mydriasis during intraocular surgery, treatment of bronchospasm, sensitivity reactions, cardiac arrhythmias, GI and renal hemorrhage, superficial bleeding, premature labor, hypoglycemia, and cardiogenic, hemorrhagic, and traumatic shock. Epinephrine can also be used to increase blood flow in ACLS during CPR, as an adjunct to local anaesthesia, and for radiographic uses.

Allergic emergencies, such as anaphylaxis, are a growing concern, given the increasing awareness of members of the public of their frequency and potential severity. Anaphylaxis is a sudden, severe, systemic allergic reaction that can be fatal, in many cases, if left untreated. Anaphylaxis can involve various areas of the body, such as the skin, respiratory tract, gastrointestinal tract, and cardiovascular system. Acute symptoms occur from within minutes to two hours after contact with the allergy-causing substance, but in rare instances onset may be delayed by as much as four hours. Contact with anaphylaxis-inducing agents, and the severity of the resulting anaphylactic reaction, can be extremely unpredictable. Accordingly, allergists recommend that persons who have a personal or family history of anaphylaxis be prepared to self-administer emergency treatment at all times. Additionally, adults charged with caring for children who are at risk for anaphylaxis should also be prepared to administer anti-anaphylactic first aid.

The symptoms of anaphylaxis include one or more of the following, generally within 1 to about 15 minutes of exposure to the antigen: agitation, a feeling of uneasiness, flushing, palpitations, paresthesias, pruritus, throbbing in the ears, coughing, sneezing, urticaria, angioedema, difficulty breathing due to laryngeal edema or bronchospasm, nausea, vomiting, abdominal pain, diarrhoea, shock, convulsions, incontinence, unresponsiveness and death. An anaphylactic reaction may include cardiovascular collapse, even in the absence of respiratory symptoms.

Epinephrine is available in a variety of formulations suited for different clinical indications and routes of administration, for example by injection, by inhalation, or topically. Its uses include at least the following: combating low blood pressure during hemorrhagic or allergic shock, opening the airways during asthmatic attack, restricting the distribution of locally administered drugs such as local anesthetics, reducing nasal congestion; and/or performance aid in emergency situations.

Epinephrine can be prepared synthetically by one of several processes readily available to one in the art. One such process starts with 1,2-dihydroxybenzene that is converted successively to (chloroacetyl)catechol with chloroacetyl chloride, then to (methyl-amino acetyl) catechol with methylamine and to racemic epinephrine by hydrogenation. The racemic form is resolved with D-tartaric acid to provide a white to nearly-white powder that is sensitive to light, air, heat, or alkaline conditions. Salts with acids are readily formed and provide some stability.

The hydrochloride, sulphate, and bitartrate salts are known in the art.

The racemization and oxidation are the two major problems associated with liquid formulations of epinephrine which may result in reduced potency, less desirable effects, or have the potential to cause harm. Racemization of l-epinephrine into its less biologically active dextrorotatory isoform, d-epinephrine, is undesirable because of its significantly low pressor effect; about one-fifteenth that of l-epinephrine. The d-isoform may also affect adrenergic receptor subtypes differently than the l-isoform, resulting in substandard and undesirable effects. Oxidation of epinephrine's alcohol group forms its less potent ketone form, known as adrenalone, which has little if any beta-adrenergic activity. Thus, both racemization and oxidation of epinephrine are associated with reduced potency and less desirable effects as the impurities d-epinephrine and adrenalone form at the expense of l-epinephrine.

To deal with the problem of oxidation, the prior art teaches addition of bisulfite antioxidants and increasing overages in formulation; however both of these have the potential to cause harm to patients. Antioxidants, such as sodium metabisulfite, are added to epinephrine formulations to reduce oxidation and to help keep formulations sterile. Sterilization techniques themselves often result in the loss of total epinephrine, and l-epinephrine, which may be compensated with increased overages. Sodium bisulfite and sodium metabisulfite, bisulfites, can cause mild to severe, life-threatening allergic reactions, including anaphylaxis or asthmatic episodes in susceptible individuals, especially those with sulfite sensitivities. So, while epinephrine is indicated for treating anaphylaxis, the presence of sulfites in its formulation puts susceptible patients at great risk of exacerbating their anaphylaxis to the point of death. And for patients who are in other critical situations, such as cardiac arrest or septic shock, such sulfite reactions could greatly worsen the critical condition of these vulnerable patients. Most formulations also use overages of active pharmaceutical ingredient to compensate for degradation of epinephrine content and activity over the course of the product's shelf-life. This results in epinephrine drug products released after manufacturing with a higher than expected activity, which could be hazardous to patients as causing higher infusion and injection doses, thereby increasing side effects such as tachycardia.

In addition to the degradants d-epinephrine and adrenalone, which have been mentioned to have little pharmacological activity compared with l-epinephrine, lesser other degradants include adrenochrome and adrenolotin. A potentially toxic impurity, epinephrine sulfonate, forms by sulfonation reaction in epinephrine drug products containing sulfites.

Epinephrine injection, 1 mg/mL for intramuscular, subcutaneous, and intravenous use, is currently approved in the U. S. Food and Drug Administration (FDA) under the brand name Adrenalin® as 1 mL single dose vial and 30 mL multi-dose vial by Par Sterile Products LLC. The product is indicated for anaphylaxis and hypotension associated with septic shock. The 1 mL vial of Adrenalin solution contains 1 mg epinephrine, 7.3 mg sodium chloride, 0.457 mg sodium metabisulfite, 1 mg sodium hydroxide, 2.25 mg tartaric acid, 0.20 mg disodium edetate dihydrate, hydrochloric acid to adjust pH, and water for injection. In the 30 mL vial, each 1 mL of Adrenalin solution contains 1 mg epinephrine, 6.15 mg sodium chloride, 0.457 mg sodium metabisulfite, 0.920 mg sodium hydroxide, 2.25 mg tartaric acid, 0.20 mg disodium edetate dihydrate, hydrochloric acid to adjust pH, 5.25 mg chlorobutanol as a preservative and water for injection. The pH range is 2.2-5.0.

The FDA also approved Belcher's Epinephrine injection 1 mg/mL, indicated for increase of mean arterial blood pressure in adult patients with hypotension associated with septic shock and for induction and maintenance of mydriasis during intraocular surgery. Each mL contains 1 mg Epinephrine base (as the hydrochloride), Sodium Chloride 9 mg (for isotonicity), Water for Injection, USP, and HCl to adjust pH. The product contains no sulfites.

In the field of injectable preparations, liquid epinephrine injection for intramuscular, subcutaneous, and intravenous administration have already been described in scientific and patent literature.

FDA originally approved Adrenalin® 1 mL with 18-month shelf life and each mL contains 1 mg epinephrine, 9.0 mg sodium chloride, 1.0 mg sodium metabisulfite, HCl to adjust pH, and water for injection and the 30 mL product approved for an even shorter shelf life of 14 months, with each mL contains 1 mg epinephrine, 9.0 mg sodium chloride, 1.5 mg sodium metabisulfite, hydrochloric acid to adjust pH, 5.4 mg chlorobutanol as a preservative and water for injection. Shelf life is limited by the formation of degradants, which mainly comprise epinephrine sulfonic acid (ESA) and D-epinephrine, an enantiomer of L-epinephrine that has insignificant therapeutic activity.

U.S. Pat. No. 9,119,876 B1 and its equivalents disclose stabilized epinephrine pharmaceutical compositions and their use, wherein the compositions comprise sodium bisulfite and/or sodium metabisulfite as antioxidant and tartaric acid. The composition according to invention is characterized as slowing the rate of epinephrine degradation (epinephrine sulfonic acid, total related substance and d-epinephrine) as compared to prior art formulations of Adrenalin®.

U.S. Pat. No. 10,004,700 B1 and its equivalents disclose pharmaceutical formulations of levorotatory-epinephrine which is more potent and less toxic than existing pharmaceutical formulations of epinephrine, wherein the compositions have a pH between 2.8 and 3.3.

U.S. Patent Publication No. US 2008/0269347 A1 discloses injectable pharmaceutical composition comprising epinephrine, EDTA, and at least one antioxidant, wherein the antioxidant is selected from the group consisting of cysteine, citric acid, thioglycerol, acetylcysteine, and a combination thereof. The compositions are characterized as being significantly better than metabisulfite-containing formulations.

PCT Publication No. WO2014127018 A1 discloses pharmaceutical formulations comprising epinephrine or its salt thereof, hydroxyl propyl β-cyclodextrin, and a tonicity modifier, in an aqueous solution.

The present Adrenalin injection composition contains sodium metabisulfite as an antioxidant. The presence of sodium metabisulfite in the formulation may increases the chance of racemization and sulfonation by reacting with the epinephrine. To overcome the racemization and sulfonation issues, the inventors of the present invention have developed epinephrine formulations by using an antioxidant that may prevent or inhibit the sulfonation and racemization of epinephrine. In particular, the inventors of the present invention have developed a liquid formulation comprising levorotatory-epinephrine (l-epinephrine) in a concentration of about 0.5 to 1.5 mg/mL and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or combination. thereof. The present invention also relates to a process of preparing such compositions and use thereof.

### SUMMARY OF THE INVENTION

The present invention provides liquid formulations of epinephrine or a pharmaceutically acceptable salt thereof intended for parenteral administration. In particular, the invention provides a liquid formulation comprising levorotatory-epinephrine (l-epinephrine) in a concentration of about 0.5 to 1.5 mg/mL and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or combination.

In one general aspect, there is provided a liquid formulation of epinephrine or a pharmaceutically acceptable salt thereof in a concentration of about 0.5 to 1.5 mg/mL and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof.

In one general aspect, there is provided a liquid formulation of l-epinephrine or a pharmaceutically acceptable salt thereof in a concentration of about 0.5 to 1.5 mg/mL and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof.

In one general aspect, there is provided a liquid formulation of l-epinephrine or a pharmaceutically acceptable salt thereof in a concentration of about 0.5 to 1.5 mg/mL, citric acid and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof.

In one general aspect, there is provided a liquid formulation of l-epinephrine in a concentration of about 0.5 to 1.5 mg/mL, citric acid and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof.

In one general aspect, there is provided a liquid formulation of l-epinephrine in a concentration of about 0.5 to 1.5 mg/mL, a buffering agent, a chelating agent, a tonicity regulating agent, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof.

In one general aspect, there is provided a liquid formulation of l-epinephrine in a concentration of about 0.5 to 1.5 mg/mL, a buffering agent, a chelating agent, a tonicity regulating agent, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to 5.0 mg/mL.

In one general aspect, there is provided a liquid formulation of l-epinephrine in a concentration of about 0.5 to 1.5 mg/mL, a buffering agent, a chelating agent, a tonicity regulating agent, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof.

In one general aspect, there is provided a liquid formulation of l-epinephrine in a concentration of about 0.5 to 1.5 mg/mL, a buffering agent, a chelating agent, a tonicity regulating agent, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to 5.0 mg/mL.

In one general aspect, there is provided a liquid formulation of l-epinephrine in a concentration of about 0.5 to 1.5 mg/mL, citric acid, sodium edetate, sodium chloride, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to 5.0 mg/mL.

In one general aspect, there is provided a liquid formulation of l-epinephrine in a concentration of about 1.0 mg/mL, citric acid, sodium edetate, sodium chloride, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to 5.0 mg/mL.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to 0.5 mg/mL, sodium chloride in a concentration of about 4.0 to about 9.0 mg/mL water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to 5.0 mg/mL.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to 0.5 mg/mL, sodium chloride in a concentration of about 4.0 to about 9.0 mg/mL water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to 5.0 mg/mL and the composition pH is range of about 2 to 5.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to about 5.0 mg/mL and the composition is free of bisulfite.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to about 5.0 mg/mL and the composition's final volume may be 1 mL or 30 mL.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to about 5.0 mg/mL and the composition is suitable for subcutaneous, intracameral, intravenous, and intramuscular injection, infusion, intra-arterial administration, intracardiac injection, endotracheal administration, intraosseous administration, oral inhalation, topical administration, and as ophthalmic irrigation.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to 0.5 mg/mL, sodium chloride in a concentration of about 4.0 to about 9.0 mg/mL, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to about 5.0 mg/mL, which may limit the formation of oxidative degradants in the composition to less than about 1%, after a certain period of shelf life.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to about 5.0 mg/mL and the composition filled into, but were not limited to, glass ampules, glass vials with caps, glass bottles with caps, and syringes to make prefilled syringes or autoinjectors.

In one general aspect, there is provided a process for preparation of a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to about 5.0 mg/mL.

In one general aspect, there is provided a process for preparation of a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to about 5.0 mg/mL and the composition is filled in a vial, an ampoule, a bag, a bottle, a cartridge, or a syringe.

In another general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to about 5.0 mg/mL and characterized in that the formulation retains at least 90% w/w of the potency of epinephrine when stored at 25° C. and 60% relative humidity or at 40° C. and 75% relative humidity for 3 months.

In another general aspect, there is provided a process for preparing a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to 5.0 mg/mL, wherein the obtained formulation is aseptically distributed into single dose or multidose containers.

In another general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is selected from the group consisting of ascorbic acid, sodium ascorbate, monothioglycerol, vitamin-A, vitamin-B₁, vitamin-B₂, vitamin-B₆, vitamin-E, butylated hydroxy toluene, butylated hydroxy anisole and propyl gallate, alone or in a combination thereof in a concentration of about 0.2 to about 5.0 mg/mL, wherein the obtained formulation exhibits good stability throughout the shelf life as the impurities observed are well below the specified limits.

In one general aspect, there is provided a liquid formulation of l-epinephrine in a concentration of about 0.5 to about 1.5 mg/mL, a buffering agent, a chelating agent, a tonicity regulating agent, water and at least one antioxidant, wherein the antioxidant is ascorbic acid, salts or its derivatives.

In one general aspect, there is provided a liquid formulation of l-epinephrine in a concentration of about 0.5 to about 1.5 mg/mL, citric acid, sodium edetate, sodium chloride, water and at least one antioxidant, wherein the antioxidant is ascorbic acid, salts or its derivatives.

In one general aspect, there is provided a liquid formulation of l-epinephrine in a concentration of about 1.0 mg/mL, citric acid, sodium edetate, sodium chloride, water and at least one antioxidant, wherein the antioxidant is ascorbic acid, salts or its derivatives.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL water and at least one antioxidant, wherein the antioxidant is ascorbic acid, salts or its derivatives in a concentration of about 0.2 to about 5.0 mg/mL.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL water and at least one antioxidant, wherein the antioxidant is ascorbic acid in a concentration of about 0.2 to about 5.0 mg/mL and the composition pH is range of about 2 to 5.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL water and at least one antioxidant, wherein the antioxidant is ascorbic acid in a concentration of about 0.2 to about 5.0 mg/mL and the composition is free of bisulfite.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL water and at least one antioxidant, wherein the antioxidant is ascorbic acid in a concentration of about 0.2 to about 5.0 mg/mL and the composition's final volume may be 1 mL or 30 mL.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL water and at least one antioxidant, wherein the antioxidant is ascorbic acid in a concentration of about 0.2 to about 5.0 mg/mL and the composition is suitable for subcutaneous, intracameral, intravenous, and intramuscular injection, infusion, intraarterial administration, intracardiac injection, endotracheal administration, intraosseous administration, oral inhalation, topical administration, and as ophthalmic irrigation.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is ascorbic acid in a concentration of about 0.2 to about 5.0 mg/mL which may limit the formation of oxidative degradants in the composition to less than about 1%, after a certain period of shelf life.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is ascorbic acid in a concentration of about 0.2 to about 5.0 mg/mL and the composition is filled into, but were not limited to, glass ampules, glass vials with caps, glass bottles with caps, and syringes to make prefilled syringes or autoinjectors.

In one general aspect, there is provided a process for preparation of a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is ascorbic acid, salts or its derivatives in a concentration of about 0.2 to about 5.0 mg/mL.

In one general aspect, there is provided a process for preparation of a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is ascorbic acid, salts or its derivatives and the composition is filled in a vial, an ampoule, a bag, a bottle, a cartridge, or a syringe.

In another general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is ascorbic acid, salts or its derivatives and characterized in that the formulation retains at least 90% w/w of the potency of epinephrine when stored at 25° C. and 60% relative humidity or at 40° C. and 75% relative humidity for 3 months.

In another general aspect, there is provided a process for preparing a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is ascorbic acid, salts or its derivatives, and wherein the obtained formulation is aseptically distributed into single dose or multidose containers.

In another general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is ascorbic acid, salts or its derivatives, and wherein the obtained formulation exhibits good stability throughout the shelf life as the impurities observed are well below the specified limits.

In one general aspect, there is provided a liquid formulation of l-epinephrine in a concentration of about 0.5 to about 1.5 mg/mL, a buffering agent, a chelating agent, a tonicity regulating agent, water and at least one antioxidant, wherein the antioxidant is monothioglycerol, salts or its derivatives.

In one general aspect, there is provided a liquid formulation of l-epinephrine in a concentration of about 0.5 to about 1.5 mg/mL, citric acid, sodium edetate, sodium chloride, water and at least one antioxidant, wherein the antioxidant is monothioglycerol, salts or its derivatives.

In one general aspect, there is provided a liquid formulation of l-epinephrine in a concentration of about 1.0 mg/mL, citric acid, sodium edetate, sodium chloride, water and at least one antioxidant, wherein the antioxidant is monothioglycerol, salts or its derivatives.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol, salts or its derivatives in a concentration of about 1.0 to about 5.0 mg/mL.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol in a concentration of about 1.0 to about 5.0 mg/mL and the composition pH is in a range of about 2 to about 5.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol in a concentration of about 1.0 to about 5.0 mg/mL and the composition is free of bisulfite.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol in a concentration of about 1.0 to about 5.0 mg/mL and the composition final volume may be 1 mL or 30 mL.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol in a concentration of about 1.0 to about 5.0 mg/mL and the composition is suitable for subcutaneous, intracameral, intravenous, and intramuscular injection, infusion, intra-arterial administration, intracardiac injection, endotracheal administration, intraosseous administration, oral inhalation, topical administration, and as ophthalmic irrigation.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol in a concentration of about 1.0 to about 5.0 mg/mL which may limit the formation of oxidative degradants in the composition to less than about 1%, after a certain period of shelf life.

In one general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol in a concentration of about 1.0 to about 5.0 mg/mL and the composition is filled into, but were not limited to, glass ampules, glass vials with caps, glass bottles with caps, and syringes to make prefilled syringes or autoinjectors.

In one general aspect, there is provided a liquid formulation of l-epinephrine in a concentration of about 0.5 to about 1.5 mg/mL, tartaric acid, sodium edetate, sodium chloride and water, wherein the composition is free of bisulfite antioxidant.

In one general aspect, there is provided a liquid formulation of l-epinephrine in a concentration of about 1.0 mg/mL, tartaric acid in a concentration of about 0.1 to about 4.0 mg/mL, sodium edetate in a concentration of about 0.1 to about 0.5 mg/mL, sodium chloride in a concentration of about 4.0 to about 9.0 mg/mL and water, wherein the composition is free of bisulfite antioxidant and composition has a pH in the range of about 2.5 to about 3.5.

In one general aspect, there is provided a liquid formulation of l-epinephrine in a concentration of about 1.0 mg/mL, tartaric acid in a concentration of about 0.1 to about 4.0 mg/mL, sodium edetate in a concentration of about 0.1 to about 0.5 mg/mL, sodium chloride in a concentration of about 4.0 to about 9.0 mg/mL and water, wherein the composition is free of bisulfite antioxidant and the composition comprises about 10% or less total impurities after 3 months of storage at about 25° C. and 60% RH and or about 40° C. and 75% RH.

In one general aspect, there is provided a liquid formulation of l-epinephrine in a concentration of about 1.0 mg/mL, tartaric acid in a concentration of about 0.1 to about 4.0 mg/mL, sodium edetate in a concentration of about 0.1 to about 0.5 mg/mL, sodium chloride in a concentration of about 4.0 to about 9.0 mg/mL and water, wherein the composition is free of bisulfite antioxidant and the composition is filled into, but not limited to, glass ampules, glass vials with caps, glass bottles with caps, and syringes to make prefilled syringes or autoinjectors.

In one general aspect, there is provided a process for preparing a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol, salts or its derivatives in a concentration of about 1.0 to about 5.0 mg/mL.

In one general aspect, there is provided a process for preparing a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol, salts or its derivatives and the composition is filled in a vial, an ampoule, a bag, a bottle, a cartridge, or a syringe.

In another general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol, salts or its derivatives and characterized in that the formulation retains at least 90% w/w of the potency of epinephrine when stored at 25° C. and 60% relative humidity or at 40° C. and 75% relative humidity for 3 months.

In another general aspect, there is provided a process for preparing a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol, salts or its derivatives, and wherein the obtained formulation is aseptically distributed into single dose or multidose containers.

In another general aspect, there is provided a process for preparing a liquid formulation of 1-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL and water, wherein the composition is free of bisulfite antioxidant and the composition has a pH in the range of about 2.5 to about 3.5.

In another general aspect, there is provided a liquid formulation of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, water and at least one antioxidant, wherein the antioxidant is monothioglycerol, salts or its derivatives, and wherein the obtained formulation exhibits good stability throughout the shelf life as the impurities observed are well below the specified limits.

### DETAILED DESCRIPTION OF THE INVENTION

The invention provides liquid formulations of epinephrine or a pharmaceutically acceptable salt thereof intended for parenteral administration. In particular, the invention provides a liquid formulation comprising levorotatory-epinephrine (l-epinephrine) in a concentration of about 0.5 to 1.5 mg/mL and at least one antioxidant, wherein the antioxidant is selected from the group consisting of ascorbic acid, monothioglycerol and a combination thereof.

The term “epinephrine” used throughout the specification refers to not only epinephrine per se, but also its other pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof. Examples of epinephrine salts include, but are not limited to, hydrochloride, sulphate, and bitartrate.

The term “formulation” as used herein, is interchangeable with composition and refers to preparations comprising epinephrine or a pharmaceutically acceptable salt; in a form suitable for administration to a mammal.

The term “stable” as used herein, refers to any preparation of epinephrine or pharmaceutically acceptable salts thereof having sufficient physical and chemical stability to allow for storage at a convenient temperature, such as between about 0° C. and about 50° C., for a commercially reasonable period of time. The term “physical stability” refers to maintenance of colour, dissolved oxygen level, head space oxygen level, and particulate matter. The term “chemical stability” relates to formation of drug-related impurities in terms of total impurity, single maximum individual impurity and maximum individual unknown impurity. For the purpose of the present invention chemical stability also includes maintenance of pH of the finished formulation. For pharmaceutical products, stability is required for commercially relevant times after manufacturing, such as for about 6, 12, 18, 24 or 36 months, during which a product is kept in its original packaging under specified storage condition.

The term “shelf life” refers to the amount of time the formulations may be stored without loss of potency and/or dissolution profile. Preferably, the shelf life refers to the amount of time the formulations may be stored without a loss of more than 2%, 5%, 8% or 10% of the potency and/or dissolution.

The term “about” as used herein, is used where measurements are understood to vary due to measurement issues or variability in populations, such as results of clinical studies. The scope of such terms will depend on the context of the element at issue and the understanding of those skilled in the art. In the absence of such guidance in the art, through relevant teachings or examples, the term “about” should be understood as meaning +/−10% of the indicated value(s).

The term “impurity” refers to an undesired substance in a composition which may be present in an initial composition and/or may be formed after a certain period of shelf life of a composition. These impurities may be formed via degradation of one or more components of the composition. Sources of degradation include, but are not limited to, oxidation, racemization, sulfite addition, visible light, ultraviolet light, moisture, heat, changes in pH, and composition component interactions.

The stable epinephrine formulation refers to a formulation that retains at least about 90%, or about least about 95%, or at least about 96%, or at least about 98%, of the labelled concentration of epinephrine or pharmaceutically acceptable salt thereof after storage under typical and/or accelerated conditions.

The concentration of epinephrine or a pharmaceutically acceptable salt in the formulation is present at a concentration of about 0.1 to 2 mg/mL, preferably about 0.5 to 1.5 mg/mL, more preferably about 0.75 to 1.25 mg/mL, and most preferably about 1 mg/mL. In some embodiments, the active agent is present at a concentration of 1.14 mg/mL.

The formulations include epinephrine or a pharmaceutically acceptable salt suitable for parenteral administration and one or more pharmaceutically acceptable excipients selected from buffers or buffering agents, chelating agents, antioxidants, stabilizing agents, tonicity agents, pH adjusting agents, preservatives and water.

The antioxidant used in the epinephrine formulation may prevent and/or inhibit the formation of unacceptable amounts of oxidative degradants in the formulation after a certain period of shelf life. These antioxidants may prevent the sulfonation and racemization of epinephrine. Exemplary antioxidants include, but are not limited to ascorbic acid, sodium ascorbate, monothioglycerol, vitamin-A, vitamin-B₁, vitamin-B₂, vitamin-B₆, vitamin-E, butylated hydroxy toluene, butylated hydroxy anisole, dithioerythreitol, propyl gallate and thioglycolic acid, alone or in combinations thereof. The antioxidant may be present at a concentration in the range of about 0.2 to about 10 mg/mL, preferably in the range of about 0.2 to about 5.0 mg/mL.

The buffering agent used in epinephrine formulation may provide a resistance to significant change in pH caused by a strong acid or base by interacting with a strong acid or strong base in a formulation or solution. The buffering agent may comprise a single agent or more than one agent, such as a weak acid and its conjugate base. Exemplary buffers include, but are not limited to citrate, glutamate, lactate, malate, gluconate, benzoate, succinate, acetate, glycine, and aspartate, alone or in combinations thereof. The buffering agent may have a buffer range from a pH of about 2 to 5, preferably from a pH of about 3 to 4.5, and more preferably from a pH of about 3.5 to 4.5.

The pH-adjusting agent(s) are used in the liquid formulations to adjust the pH to a desirable range. Exemplary pH-adjusting agents are well known by those skilled in the art and include sodium hydroxide, sodium carbonate, sodium bicarbonate, potassium hydroxide, ammonium carbonate, hydrochloric acid, citric acid, tartaric acid, lactic acid, phosphoric acid, sodium phosphate, sulfuric acid and the like. While not intending to limit the scope of the invention in any way, certain formulations disclosed herein may have a pH of about 2 to 5, preferably from a pH of about 3 to 4.5, and more preferably from a pH of about 3.0 to 3.5.

The tonicity agents are used in liquid formulations to adjust the composition of the formulation to be within the desired isotonic range. Exemplary tonicity agents include, but are not limited to sodium chloride, potassium chloride, dextrose, glycerol, and mannitol. The tonicity agents are in the amount of about 0.01% to about 10% by weight.

Chelating agents are used in the epinephrine formulations to enhance preservative effectiveness by forming stable water-soluble complexes (chelates) with alkaline earth and heavy metal ions. Exemplary chelating agents include, but are not limited to, ethylenediaminetetraacetic acid (EDTA), or salts thereof. The chelating agent typically is present in an amount from about 0.001-0.1% by weight. In the case of EDTA, the chelating agent is preferably present at a concentration of about 0.025% by weight.

The preservatives in the liquid formulations are used to inhibit microbial growth. Suitable preservatives include, but are not limited to, benzalkonium chloride, benzethonium chloride, benzoic acid, benzyl alcohol, benzyl paraben, bronopol, butyl paraben, cetrimide, cetylpyridinium chloride, chlorobutanol, chlorhexidine, chlorocresol, chloroxylenol, cresol, ethyl alcohol, ethyl paraben, ethylparaben, glycerin, hexetidine, imidurea, isobutyl paraben, meta-cresol, methyl paraben, methylparaben, phenol, phenoxyethanol, phenylethyl alcohol, phenylmercuric nitrate, p-hydroxybenzoic acid esters, polyhexamethylene biguanide, potassium sorbate, propyl paraben, propylene glycol, sodium benzoate, sodium perborate, sodium propionate, sorbic acid, stabilized thimerosal, and/or thimerosal and the like. The preservative may be present in the composition at a concentration in the range of about 1 to 10 mg/mL.

The formulations can be prepared by using any suitable technique, many of which are known to those skilled in the art and can be combined in any order.

The formulations disclosed herein can consist or consist essentially of the listed ingredients. If the formulations consist essentially of the listed ingredients, other ingredients may be present so long as they do not affect the stability of the epinephrine in the formulation, for example, as measured by assay.

It is to be understood that the above description is intended to be illustrative and not restrictive. Many embodiments will be apparent to those of skill in the art upon reading the above description. The scope of the invention should, therefore, be determined with reference to the appended claims, along with the full scope of equivalents to which such claims are entitled. The disclosures of all articles and references, including patents, patent applications and publications, are incorporated herein by reference in their entirety and for all purposes.

### Example 1: Epinephrine Injection Composition with Ascorbic Acid and Citric Acid

| Sr. No | Ingredient | Quantity/mL |
|---|---|---|
| 1 | L-Epinephrine | 0.5 mg-1.5 mg |
| 2 | Sodium chloride | 7.3 mg |
| 3 | Ascorbic acid | 0.2 mg-5.0 mg |
| 4 | Sodium Hydroxide | 0.5 mg |
| 5 | Citric acid monohydrate | 1.5 mg |
| 6 | Disodium edetate dihydrate | 0.20 mg |
| 7 | Hydrochloric acid | Qs to adjust the pH |
| 8 | Water | Qs to required volume |

Manufacturing Process: The manufacturing process has the following steps:
1. In a suitable container, take 80% of the required water and bring it to 25° C., under nitrogen purging.
2. Add EDTA, citric acid and sodium chloride, one after another, making sure that the previous component has dissolved before the addition of the next component.
3. Add the ascorbic acid.
4. Add the weighed quantity of L-Epinephrine.
5. Mix and adjust the pH to 3.4±0.1 by using 0.1 N HCl or 0.1 N NaOH.
6. Make up the volume with water, stir for an additional 15 minutes and then transfer into appropriate sterile containers.

### Example 2: Epinephrine Injection Composition with Ascorbic Acid and Citric Acid

| Sr. No | Ingredient | Quantity/mL |
|---|---|---|
| 1 | L-Epinephrine | 0.5 to 1.5 mg/mL |
| 2 | Chlorobutanol | 5.25 mg |
| 3 | Sodium chloride | 6.15 mg |
| 4 | Ascorbic acid | 0.2 mg-5.0 mg |
| 5 | Sodium Hydroxide | 0.5 mg |
| 6 | Citric acid monohydrate | 1.5 mg |
| 7 | Disodium edetate dihydrate | 0.20 mg |
| 8 | Hydrochloric acid | Qs to adjust the pH |
| 9 | Water | Qs to required volume |

Manufacturing Process: The manufacturing process has the following steps:
1. In a suitable container, take 80% of the required water and bring it to 25° C., under nitrogen purging.
2. Add EDTA, citric acid and sodium chloride, one after another, making sure that the previous component has dissolved before the addition of the next component.
3. Add the chlorobutanol and ascorbic acid.
4. Add the weighed quantity of L-Epinephrine.
5. Mix and adjust the pH to 3.4±0.1 by using 0.1 N HCl or 0.1 N NaOH.
6. Make up volume with water, stir for additional 15 minutes and transfer into appropriate sterile containers.

### Example 3: Epinephrine Injection Composition with Monothioglycerol and Citric Acid

| Sr. No | Ingredient | Quantity/mL |
|---|---|---|
| 1 | L-Epinephrine | 0.5 to 1.5 mg/mL |
| 2 | Sodium chloride | 7.3 mg |
| 3 | Monothioglycerol | 1.0 mg-5.0 mg |
| 4 | Sodium Hydroxide | 0.5 mg |
| 5 | Citric acid monohydrate | 1.5 mg |
| 6 | Disodium edetate dihydrate | 0.20 mg |
| 7 | Hydrochloric acid | Qs to adjust the pH |
| 8 | Water | Qs to required volume |

Manufacturing Process: The manufacturing process has the following steps:
1. In a suitable container, take 80% of the required water and bring it to 25° C., under nitrogen purging.
2. Add EDTA, citric acid and sodium chloride, one after another, making sure that the previous component has dissolved before the addition of the next component.
3. Add the monothioglycerol.
4. Add the weighed quantity of L-Epinephrine.
5. Mix and adjust the pH to 3.4±0.1 by using 0.1 N HCl or 0.1 N NaOH.
6. Make up volume with water, stir for an additional 15 minutes and transfer into appropriate sterile containers.

### Example 4: Epinephrine Injection Composition with Monothioglycerol and Citric Acid

| Sr. No | Ingredient | Quantity/mL |
|---|---|---|
| 1 | L-Epinephrine | 0.5 to 1.5 mg/mL |
| 2 | Chlorobutanol | 5.25 mg |
| 3 | Sodium chloride | 6.15 mg |
| 4 | Monothioglycerol | 1.0 mg-5.0 mg |
| 5 | Sodium Hydroxide | 0.5 mg |
| 6 | Citric acid monohydrate | 1.5 mg |
| 7 | Disodium edetate dihydrate | 0.20 mg |
| 8 | Hydrochloric acid | Qs to adjust the pH |
| 9 | Water | Qs to required volume |

Manufacturing Process: The manufacturing process has the following steps:
1. In a suitable container, take 80% of the required water and bring it to 25° C., under nitrogen purging.
2. Add EDTA, citric acid and sodium chloride, one after another, making sure that the previous component has dissolved before the addition of the next component.
3. Add the chlorobutanol and monothioglycerol.
4. Add weighed quantity of L-Epinephrine.
5. Mix and adjust the pH to 3.4±0.1 by using 0.1 N HCl or 0.1 N NaOH.
6. Make up the volume with water, stir for an additional 15 minutes and transfer into appropriate sterile containers.

### Example 5: Epinephrine Injection Composition with Sodium Edetate Dihydrate, Tartaric Acid and Free of Bisulfite (Single Dose)

| Sr. No | Ingredient | Quantity/mL |
|---|---|---|
| 1 | L-Epinephrine | 0.5 to 1.5 mg/mL |
| 2 | Sodium chloride | 7.3 mg |
| 3 | Disodium edetate dihydrate | 0.20 mg |
| 4 | Sodium Hydroxide | 1.0 mg |
| 5 | Tartaric acid | 2.25 mg |
| 6 | Hydrochloric acid | Qs to adjust the pH 2.5-3.5 |
| 7 | Water | Qs to required volume |

Manufacturing Process: The manufacturing process has the following steps:
1. In a suitable container, take 85% of the required water and bring it to 25° C., under nitrogen purging.
2. Add tartaric acid, sodium hydroxide, sodium chloride and disodium edetate dihydrate, one after another, making sure that the previous component has dissolved before the addition of the next component.
3. Add weighed quantity of L-Epinephrine.
4. Mix and adjust the pH to 2.5 to 3.5±0.2 by using 0.1 N HCl or 0.1 N NaOH.
5. Make up the volume with water, stir for an additional 15 minutes and transfer into appropriate sterile containers.

### Example 6: Epinephrine Injection Composition with Sodium Edetate Dihydrate, Tartaric Acid, Chlorobutanol and Free of Bisulfite (Multi-Dose)

| Sr. No | Ingredient | Quantity/mL |
|---|---|---|
| 1 | L-Epinephrine | 0.5 to 1.5 mg/mL |
| 2 | Chlorobutanol | 5.25 mg |
| 3 | Sodium chloride | 7.3 mg |
| 4 | Disodium edetate dihydrate | 0.20 mg |
| 5 | Sodium Hydroxide | 0.920 mg |
| 6 | Tartaric acid | 2.25 mg |
| 7 | Hydrochloric acid | Qs to adjust the pH 2.5-3.5 |
| 8 | Water | Qs to required volume |

Manufacturing Process: The manufacturing process has the following steps:
1. In a suitable container, take 85% of the required water and bring it to 25° C., under nitrogen purging.
2. Add tartaric acid, sodium hydroxide, sodium chloride and disodium edetate dihydrate, one after another, making sure that the previous component has dissolved before the addition of the next component.
3. Add weighed quantity of L-Epinephrine.
4. Mix and adjust the pH to 2.5 to 3.5±0.2 by using 0.1 N HCl or 0.1 N NaOH.
5. Add weighed quantity of Chlorobutanol.
6. Make up the volume with water, stir for an additional 15 minutes and transfer into appropriate sterile containers.

The formulations described herein are tested for stability including one or more of assay for label claim of l-epinephrine, pH, osmolality, sterility, particulate matter, and antimicrobial preservative efficacy. The stability conditions include 25° C./60% relative humidity, 40° C./75% relative humidity, 25° C./40% relative humidity, and/or 40° C./25% relative humidity. The formulations of Examples 5 and 6, adjusted to a pH range of about 2.5 to 3.5 and charged for stability testing and the analytical results are summarized in the tables below. It was observed from stability studies that the formulations (pH range of about 2.5 to 3.5) are stable and no significant increase in impurities were observed even at accelerated conditions. The formulations of Examples 5 and 6 were found to be stable when the containers were stored inverted or oriented up.

TABLE 1
Stability data for Example-5 (pH 2.5)

| | |
|---|---|
| 40° C./75% RH | 25° C./60% RH |

| | | | | | | | | | |
|---|---|---|---|---|---|---|---|---|---|
| Test Parameters | Initial | 1M (Inv.) | 1M (Up.) | 2M (Inv.) | 2M (Up.) | 3M (Inv.) | 3M (Up.) | 3M (Inv.) | 3M (Up.) |

| | |
|---|---|
| Description | Clear colorless solution free from visible extraneous matter. |

Related Compounds (%)

| Norepinephrine | ND | | ND | | ND | | ND | | ND | | ND | | ND | | ND | | ND | |
|---|---|---|---|---|---|---|---|---|---|---|---|---|---|---|---|---|---|---|
| Epinine | ND | | ND | | ND | | ND | | ND | | ND | | ND | | ND | | ND | |
| Oxedrine | ND | | ND | | ND | | ND | | ND | | ND | | ND | | ND | | ND | |
| Adrenalone | ND | | ND | | ND | | ND | | ND | | ND | | ND | | ND | | ND | |
| Methoxy analog | 0.045 | | 0.031 | | 0.036 | | ND | | 0.026 | | 0.033 | | 0.035 | | 0.036 | | 0.032 | |
| Impurity- F | 0.111 | | 0.089 | | 0.089 | | 0.076 | | 0.076 | | 0.090 | | 0.089 | | 0.089 | | 0.088 | |
| N-Benzyl | ND | | ND | | ND | | ND | | ND | | ND | | ND | | ND | | ND | |
| epinephrine | | | | | | | | | | | | | | | | | | |
| N-Benzyl | ND | | ND | | ND | | ND | | ND | | ND | | ND | | ND | | ND | |
| adrenalone | | | | | | | | | | | | | | | | | | |
| Any individual | % | RRT | % | RRT | % | RRT | % | RRT | % | RRT | % | RRT | % | RRT | % | RRT | % | RRT |
| unknown impurity | 0.035 | 0.92 | 0.044 | 0.92 | 0.043 | 0.920 | 0.055 | 0.92 | 0.050 | 0.92 | 0.093 | 0.92 | 0.090 | 0.92 | 0.073 | 0.92 | 0.070 | 0.92 |
| | | | | | 0.026 | 1.66 | | | | | 0.060 | 0.22 | 0.071 | 0.22 | 0.049 | 0.22 | 0.044 | 0.22 |
| Total Impurity | 0.206 | | 0.163 | | 0.194 | | 0.130 | | 0.153 | | 0.277 | | 0.284 | | 0.247 | | 0.236 | |
| pH | 2.54 | | 2.55 | | 2.50 | | 2.53 | | 2.53 | | 2.54 | | 2.54 | | 2.54 | | 2.54 | |
| Enantiomeric | 1.158 | | 2.744 | | 2.476 | | 6.380 | | 6.316 | | 12.120 | | 12.210 | | 8.923 | | 9.016 | |
| Purity (%) | | | | | | | | | | | | | | | | | | |

TABLE 2
Stability data for Example-5 (pH 3.0)

| | |
|---|---|
| 40° C./75% RH | 25° C./60% RH |

| | | | | | | | | | |
|---|---|---|---|---|---|---|---|---|---|
| Test Parameters | Initial | 1M (Inv.) | 1M (Up.) | 2M (Inv.) | 2M (Up.) | 3M (Inv.) | 3M (Up.) | 3M (Inv.) | 3M (Up.) |

| | |
|---|---|
| Description | Clear colorless solution free from visible extraneous matter. |

Related Compounds (%)

| Norepinephrine | ND | | ND | | ND | | ND | | ND | | ND | | ND | | ND | | ND | |
|---|---|---|---|---|---|---|---|---|---|---|---|---|---|---|---|---|---|---|
| Epinine | ND | | ND | | ND | | ND | | ND | | ND | | ND | | ND | | ND | |
| Oxedrine | ND | | ND | | ND | | ND | | ND | | ND | | ND | | ND | | ND | |
| Adrenalone | ND | | ND | | ND | | ND | | ND | | 0.018 | | 0.018 | | ND | | ND | |
| Methoxy analog | 0.052 | | 0.032 | | 0.036 | | 0.033 | | 0.030 | | 0.044 | | 0.042 | | 0.040 | | 0.037 | |
| Impurity- F | 0.114 | | 0.091 | | 0.091 | | 0.079 | | 0.079 | | 0.091 | | 0.090 | | 0.092 | | 0.091 | |
| N-Benzyl | ND | | ND | | ND | | ND | | ND | | ND | | ND | | ND | | ND | |
| epinephrine | | | | | | | | | | | | | | | | | | |
| N-Benzyl | ND | | ND | | ND | | ND | | ND | | ND | | ND | | ND | | ND | |
| adrenalone | | | | | | | | | | | | | | | | | | |
| Any individual | % | RRT | % | RRT | % | RRT | % | RRT | % | RRT | % | RRT | % | RRT | % | RRT | % | RRT |
| unknown impurity | 0.032 | 0.92 | 0.106 | 0.52 | 0.044 | 0.92 | 0.045 | 0.92 | 0.039 | 0.92 | 0.071 | 0.92 | 0.074 | 0.074 | 0058 | 0.92 | 0.053 | 0.92 |
| | | | | | | | | | | | 0.029 | 0.75 | 0.028 | 0.028 | 0.018 | 0.75 | 0.052 | 0.22 |
| | | | | | | | | | | | 0.089 | 0.22 | 0.083 | 0.083 | 0.053 | 0.22 | | |
| Total Impurity | 0.198 | | 0.304 | | 0.171 | | 0.157 | | 0.148 | | 0.343 | | 0.334 | | 0.262 | | 0.233 | |
| pH | 2.99 | | 2.90 | | 2.93 | | 2.98 | | 2.98 | | 2.97 | | 2.97 | | 2.96 | | 2.97 | |
| Enantiomeric | 0.926 | | 1.444 | | 1.424 | | 2.967 | | 2.962 | | 5.861 | | 5.834 | | 4.309 | | 4.304 | |
| Purity (%) | | | | | | | | | | | | | | | | | | |

TABLE 3
Stability data for Example-5 (pH 3.5)

| | | | 40° C./75% RH | | | | | | | | | | | | 25° C./60% RH | | | |
|---|---|---|---|---|---|---|---|---|---|---|---|---|---|---|---|---|---|---|
| Test Parameters | Initial | | 1M (Inv.) | | 1M (Up.) | | 2M (Inv.) | | 2M (Up.) | | 3M (Inv.) | | 3M (Up.) | | 3M (Inv.) | | 3M (Up.) | |
| Description | Clear colorless solution free from visible extraneous matter. | | | | | | | | | | | | | | | | | |
| Related Compounds (%) | | | | | | | | | | | | | | | | | | |
| Norepinephrine | ND | | ND | | ND | | ND | | 0.015 | | 0.068 | | 0.059 | | 0.028 | | 0.032 | |
| Epinine | ND | | ND | | ND | | ND | | ND | | ND | | ND | | ND | | ND | |
| Oxedrine | ND | | ND | | ND | | ND | | ND | | ND | | ND | | ND | | ND | |
| Adrenalone | ND | | ND | | ND | | ND | | ND | | 0.034 | | 0.033 | | 0.018 | | 0.020 | |
| Methoxy analog | 0.042 | | 0.034 | | 0.037 | | 0.032 | | 0.033 | | 0.055 | | 0.055 | | 0.046 | | 0.046 | |
| Impurity- F | 0.107 | | 0.087 | | 0.087 | | 0.073 | | 0.075 | | 0.087 | | 0.088 | | 0.086 | | 0.087 | |
| N-Benzyl | ND | | ND | | ND | | ND | | ND | | ND | | ND | | ND | | ND | |
| epinephrine | | | | | | | | | | | | | | | | | | |
| N-Benzyl | ND | | ND | | ND | | ND | | ND | | ND | | ND | | ND | | ND | |
| adrenalone | | | | | | | | | | | | | | | | | | |
| Any individual | % | RRT | % | RRT | % | RRT | % | RRT | % | RRT | % | RRT | % | RRT | % | RRT | % | RRT |
| unknown impurity | 0.033 | 0.92 | 0.040 | 0.92 | 0.037 | 0.92 | 0.227 | 0.52 | 0.047 | 0.92 | 0.204 | 0.22 | 0.043 | 0.30 | 0.102 | 0.22 | 0.107 | 0.22 |
| | | | | | 0.029 | 1.67 | | | | | 0.054 | 0.30 | 0.104 | 0.75 | 0.027 | 0.30 | 0.027 | 0.30 |
| | | | | | | | | | | | 0.126 | 0.75 | 0.035 | 0.88 | 0.056 | 0.75 | 0.060 | 0.75 |
| | | | | | | | | | | | 0.038 | 0.88 | 0.072 | 0.92 | 0.045 | 0.92 | 0.074 | 0.92 |
| | | | | | | | | | | | 0.051 | 0.92 | 0.022 | 0.97 | | | | |
| | | | | | | | | | | | | | 0.018 | 1.23 | | | | |
| Total Impurity | 0.182 | | 0.161 | | 0.190 | | 0.413 | | 0.214 | | 0.718 | | 0.529 | | 0.408 | | 0.454 | |
| pH | 3.51 | | 3.43 | | 3.43 | | 3.49 | | 3.49 | | 3.48 | | 3.50 | | 3.47 | | 3.49 | |
| Enantiomeric | 0.829 | | 1.104 | | 1.017 | | 1.614 | | 1.612 | | 3.071 | | 3.077 | | 2.340 | | 2.344 | |
| Purity (%) | | | | | | | | | | | | | | | | | | |

TABLE 4
Stability data for Example-6 (pH 2.5)

| | | | 40° C./75% RH | | | | | | | | | | | | 25° C./60% RH | | | |
|---|---|---|---|---|---|---|---|---|---|---|---|---|---|---|---|---|---|---|
| Test Parameters | Initial | | 1M (Inv.) | | 1M (Up.) | | 2M (Inv.) | | 2M (Up.) | | 3M (Inv.) | | 3M (Up.) | | 3M (Inv.) | | 3M (Up.) | |
| Description | Clear colorless solution free from visible extraneous matter. | | | | | | | | | | | | | | | | | |
| Related Compounds (%) | | | | | | | | | | | | | | | | | | |
| Norepinephrine | ND | | ND | | ND | | ND | | ND | | ND | | ND | | ND | | ND | |
| Epinine | ND | | ND | | ND | | ND | | ND | | ND | | ND | | ND | | ND | |
| Oxedrine | ND | | ND | | ND | | ND | | ND | | ND | | ND | | ND | | ND | |
| Adrenalone | ND | | ND | | ND | | ND | | ND | | ND | | ND | | ND | | ND | |
| Methoxy analog | 0.046 | | 0.034 | | 0.034 | | 0.023 | | ND | | 0.025 | | 0.023 | | 0.029 | | 0.034 | |
| Impurity- F | 0.110 | | 0.085 | | 0.086 | | 0.075 | | 0.075 | | 0.083 | | 0.081 | | 0.083 | | 0.083 | |
| N-Benzyl | ND | | ND | | ND | | ND | | ND | | ND | | ND | | ND | | ND | |
| epinephrine | | | | | | | | | | | | | | | | | | |
| N-Benzyl | ND | | ND | | ND | | ND | | ND | | ND | | ND | | ND | | ND | |
| adrenalone | | | | | | | | | | | | | | | | | | |
| Any individual | % | RRT | % | RRT | % | RRT | % | RRT | % | RRT | % | RRT | % | RRT | % | RRT | % | RRT |
| unknown impurity | 0.021 | 0.92 | 0.034 | 0.92 | 0.033 | 0.92 | 0.039 | 0.92 | 0.041 | 0.92 | 0.033 | 0.23 | 0.027 | 0.23 | 0.025 | 0.92 | 0.026 | 0.92 |
| | | | 0.026 | 2.70 | 0.018 | 0.28 | | | | | 0.081 | 0.92 | 0.073 | 0.92 | | | | |
| | | | 0.029 | 1.75 | | | | | | | | | | | | | | |
| | | | 0.019 | 1.66 | | | | | | | | | | | | | | |
| | | | 0.018 | 0.28 | | | | | | | | | | | | | | |
| Total Impurity | 0.178 | | 0.244 | | 0.171 | | 0.137 | | 0.116 | | 0.221 | | 0.204 | | 0.138 | | 0.143 | |
| pH | 2.50 | | 2.59 | | 2.59 | | 2.44 | | 2.43 | | 2.44 | | 2.45 | | 2.49 | | 2.48 | |
| Enantiomeric | 1.071 | | 8.093 | | 8.073 | | 11.982 | | 11.963 | | 17.469 | | 17.452 | | 17.452 | | 3.809 | |
| Purity (%) | | | | | | | | | | | | | | | | | | |

TABLE 5
Stability data for Example-6 (pH 3.0)

| | | | 40° C./75% RH | | | | | | | | | | | | 25° C./60% RH | | | |
|---|---|---|---|---|---|---|---|---|---|---|---|---|---|---|---|---|---|---|
| Test Parameters | Initial | | 1M (Inv.) | | 1M (Up.) | | 2M (Inv.) | | 2M (Up.) | | 3M (Inv.) | | 3M (Up.) | | 3M (Inv.) | | 3M (Up.) | |
| Description | Clear colorless solution free from visible extraneous matter. | | | | | | | | | | | | | | | | | |
| Related Compounds (%) | | | | | | | | | | | | | | | | | | |
| Norepinephrine | ND | | ND | | ND | | ND | | ND | | ND | | ND | | ND | | ND | |
| Epinine | ND | | ND | | ND | | ND | | ND | | ND | | ND | | ND | | ND | |
| Oxedrine | ND | | ND | | ND | | ND | | ND | | ND | | ND | | ND | | ND | |
| Adrenalone | ND | | ND | | ND | | ND | | ND | | ND | | ND | | ND | | ND | |
| Methoxy analog | 0.047 | | 0.032 | | 0.087 | | 0.032 | | 0.031 | | 0.030 | | 0.032 | | 0.035 | | 0.035 | |
| Impurity- F | 0.109 | | 0.086 | | 0.029 | | 0.073 | | 0.075 | | 0.081 | | 0.079 | | 0.084 | | 0.082 | |
| N-Benzyl | ND | | ND | | ND | | ND | | ND | | ND | | ND | | ND | | ND | |
| epinephrine | | | | | | | | | | | | | | | | | | |
| N-Benzyl | ND | | ND | | ND | | ND | | ND | | ND | | ND | | ND | | ND | |
| adrenalone | | | | | | | | | | | | | | | | | | |
| Any individual | % | RRT | % | RRT | % | RRT | % | RRT | % | RRT | % | RRT | % | RRT | % | RRT | % | RRT |
| unknown impurity | 0.028 | 0.92 | 0.027 | 0.92 | 0.030 | 0.92 | 0.037 | 0.92 | 0.054 | 0.92 | 0.054 | 0.23 | 0.057 | 0.23 | 0.025 | 0.92 | 0.030 | 0.92 |
| | | | 0.017 | 0.28 | 0.017 | 0.28 | | | | | 0.026 | 0.74 | 0.026 | 0.74 | | | | |
| | | | | | | | | | | | 0.079 | 0.92 | 0.077 | 0.92 | | | | |
| | | | | | | | | | | | | | 0.017 | 1.26 | | | | |
| Total Impurity | 0.184 | | 0.162 | | 0.163 | | 0.141 | | 0.160 | | 0.267 | | 0.287 | | 0.144 | | 0.146 | |
| pH | 3.07 | | 3.17 | | 3.16 | | 2.99 | | 2.99 | | 2.98 | | 2.98 | | 3.05 | | 3.04 | |
| Enantiomeric | 0.860 | | 3.371 | | 3.370 | | 4.668 | | 4.718 | | 7.332 | | 7.326 | | 1.762 | | 1.757 | |
| Purity (%) | | | | | | | | | | | | | | | | | | |

TABLE 6
Stability data for Example-6 (pH 3.5)

| | | | 40° C./75% RH | | | | | | | | | | | | 25° C./60% RH | | | |
|---|---|---|---|---|---|---|---|---|---|---|---|---|---|---|---|---|---|---|
| Test Parameters | Initial | | 1M (Inv.) | | 1M (Up.) | | 2M (Inv.) | | 2M (Up.) | | 3M (Inv.) | | 3M (Up.) | | 3M (Inv.) | | 3M (Up.) | |
| Description | Clear colorless solution free from visible extraneous matter. | | | | | | | | | | | | | | | | | |
| Related Compounds (%) | | | | | | | | | | | | | | | | | | |
| Norepinephrine | ND | | ND | | ND | | ND | | ND | | 0.026 | | 0.030 | | ND | | ND | |
| Epinine | ND | | ND | | ND | | ND | | ND | | ND | | ND | | ND | | ND | |
| Oxedrine | ND | | ND | | ND | | ND | | ND | | ND | | ND | | ND | | ND | |
| Adrenalone | ND | | ND | | ND | | ND | | ND | | ND | | ND | | ND | | ND | |
| Methoxy analog | 0.049 | | 0.034 | | 0.042 | | 0.031 | | 0.029 | | 0.031 | | 0.033 | | 0.032 | | 0.034 | |
| Impurity- F | 0.110 | | 0.087 | | 0.087 | | 0.074 | | 0.075 | | 0.079 | | 0.080 | | 0.081 | | 0.081 | |
| N-Benzyl | ND | | ND | | ND | | ND | | ND | | ND | | ND | | ND | | ND | |
| epinephrine | | | | | | | | | | | | | | | | | | |
| N-Benzyl | ND | | ND | | ND | | ND | | ND | | ND | | ND | | ND | | ND | |
| adrenalone | | | | | | | | | | | | | | | | | | |
| Any individual | % | RRT | % | RRT | % | RRT | % | RRT | % | RRT | % | RRT | % | RRT | % | RRT | % | RRT |
| unknown impurity | 0.025 | 0.92 | 0.031 | 0.92 | 0.036 | 0.92 | 0.050 | 0.92 | 0.053 | 0.92 | 0.115 | 0.92 | 0.133 | 0.23 | 0.025 | 0.92 | 0.030 | 0.92 |
| | | | 0.017 | 0.28 | 0.017 | 0.28 | | | | | 0.098 | 0.23 | 0.054 | 0.74 | | | | |
| | | | | | 0.017 | 2.71 | | | | | 0.045 | 0.74 | 0.024 | 0.88 | | | | |
| | | | | | | | | | | | 0.022 | 0.88 | 0.131 | 0.92 | | | | |
| | | | | | | | | | | | 0.027 | 1.26 | 0.030 | 0.126 | | | | |
| Total Impurity | 0.184 | | 0.169 | | 0.199 | | 0.172 | | 0.178 | | 0.444 | | 0.496 | | 0.141 | | 0.142 | |
| pH | 3.25 | | 3.37 | | 3.37 | | 3.16 | | 3.15 | | 3.16 | | 3.17 | | 3.23 | | 3.23 | |
| Enantiomeric | 0.824 | | 2.655 | | 2.654 | | 3.576 | | 3.581 | | 5.621 | | 5.620 | | 1.469 | | 1.467 | |
| Purity (%) | | | | | | | | | | | | | | | | | | |

The invention claimed is:

1. A liquid composition consisting essentially of levorotatory-epinephrine (l-epinephrine) in a concentration of about 0.5 to about 1.5 mg/mL, tartaric acid in a concentration of about 0.1 to about 4.0 mg/mL, sodium edetate in a concentration of about 0.1 to about 0.5 mg/mL, sodium chloride in a concentration of about 4.0 to about 9.0 mg/mL and water, wherein the composition is free of bisulfite as an antioxidant.

2. The liquid composition of claim 1, wherein the l-epinephrine is in a concentration of about 1.0 mg/mL.

3. The liquid composition of claim 1, wherein the composition has an enantiomeric purity of l-epinephrine of greater than about 92%.

4. The liquid composition of claim 1, wherein the composition is in the form of a solution for intravenous, subcutaneous or intramuscular administration.

5. The liquid composition of claim 1, wherein the composition is 1 mg/mL of l-epinephrine sterile injection solution.

6. The liquid composition of claim 1, wherein the concentration of tartaric acid is about 2.25 mg/mL, sodium edetate in a concentration of about 0.2 mg/mL, sodium chloride in a concentration of about 6.15 mg/mL and water.

7. The liquid composition of claim 1, wherein the composition further comprises chlorobutanol as preservative.

8. The liquid composition of claim 1, wherein the composition has a pH of about 2.5 to about 3.5.

9. The liquid composition of claim 1, wherein the composition is a solution filled in a vial, an ampoule, a bag, a bottle, a cartridge, or a syringe.

10. The liquid composition of claim 1, wherein the composition is stable for at least 12 months when stored at 25±2° C. and 60% relative humidity.

11. The liquid composition of claim 1, wherein the composition comprises about 10% or less total impurities after 3 months on storage at about 25° C. and 60% RH and or about 40° C. and 75% RH, wherein the impurities comprise norepinephrine, epinine, oxedrine, adrenalone, methoxy analog Impurity-F, N-benzyl epinephrine, N-benzyl adrenalone and or any other unknown impurity.

12. The liquid composition of claim 1, wherein the composition does not undergo unacceptable colour change after 3 months on storage at about 25° C. and 60% RH and or about 40° C. and 75% RH.

13. A liquid composition consisting essentially of levorotatory-epinephrine (l-epinephrine) in a concentration of about 0.5 to about 1.5 mg/mL, tartaric acid in a concentration of about 0.1 to about 4.0 mg/mL, sodium edetate in a concentration of about 0.1 to about 0.5 mg/mL, sodium chloride in a concentration of about 4.0 to about 9.0 mg/mL, optional sodium hydroxide to adjust pH, optional hydrochloric acid to adjust pH, and water, wherein the composition is free of bisulfite as an antioxidant and the composition has a pH in the range of about 2.5 to about 3.5.

14. The liquid composition of claim 13, wherein the composition consists of the l-epinephrine, tartaric acid in a concentration of about 2.25 mg/mL, sodium edetate in a concentration of about 0.2 mg/mL, sodium chloride in a concentration of about 7.3 mg/mL, the optional sodium hydroxide to adjust pH, the optional hydrochloric acid to adjust pH, and water.

15. A liquid injection composition of l-epinephrine in a concentration of about 1.0 mg/mL, citric acid in a concentration of about 0.1 to about 4.0 mg/mL, sodium edetate in a concentration of about 0.1 to about 0.5 mg/mL, sodium chloride in a concentration of about 4.0 to about 9.0 mg/mL water and at least one antioxidant, wherein the antioxidant is selected from the group consisting of ascorbic acid, sodium ascorbate, monothioglycerol, vitamin-A, vitamin-B1, vitamin-B2, vitamin-B6, vitamin-E, butylated hydroxy toluene, butylated hydroxy anisole and propyl gallate, alone or in a combination thereof, in a concentration of about 0.2 to about 5.0 mg/mL and the composition is free of bisulfite.
